(19) United States
(12) Patent Application Publication
(10) Pub. No.: US 2023/0340491 A1
(43) Pub. Date: Oct. 26, 2023

(54) COMPOSITIONS AND METHODS FOR TREATING METABOLIC AND CARDIOVASCULAR DISEASES

(71) Applicant: UNIVERSITY OF ROCHESTER, ROCHESTER, NY (US)

(72) Inventor: Zheng-Gen Jin, ROCHESTER, NY (US)

(21) Appl. No.: 17/917,358

(22) PCT Filed: Apr. 14, 2021
(86) PCT No.: PCT/US21/27182

§ 371 Date: Oct. 6, 2022

Related U.S. Application Data
(60) Provisional application No. 63/013,963, filed on Apr. 22, 2020.

(51) Int. Cl. C12N 15/113; A61K 31/506; A61P 3/04; A61P 3/06; A61P 1/16

(52) U.S. Cl. CPC C12N 15/1138; A61K 31/506; A61P 3/04; A61P 3/06; A61P 1/16

(57) ABSTRACT

Disclosed herein are novel therapeutic compositions and treatment methods for metabolic symptom and cardiovascular diseases, such as obesity, nonalcoholic fatty liver disease (NAFLD) and nonalcoholic steatohepatitis (NASH).

### PRIORITY CLAIMS AND RELATED APPLICATIONS

This application claims the benefit of priority to U.S. Provisional Application No. 63/013,963, filed Apr. 22, 2020, the entire content of which is incorporated herein by reference for all purposes.

### STATEMENT REGARDING FEDERALLY SPONSORED RESEARCH

This invention was made with government support under Grant No. HL114570, awarded by the National Institutes of Health. The Government has certain rights in the invention.

### TECHNICAL FIELD OF THE INVENTION

The invention generally relates to therapeutics and treatment methods for certain diseases and conditions. More particularly, the invention provides novel methods and compositions for treating various metabolic and cardiovascular and related diseases and conditions.

### BACKGROUND OF THE INVENTION

Metabolic syndrome (or metabolic disease) is a rising threat to human health in the United States and throughout the world. Metabolic syndrome refers to a cluster of at least three of several risk factors that may occur together, e.g., abdominal (central) obesity, high blood pressure, elevated fasting plasma glucose, high serum triglycerides, and low high-density cholesterol (HDL) levels. Despite growing recognition of the problem, the obesity epidemic continues in the U.S., and obesity rates are increasing around the world. The latest estimates are that approximately 34% of adults and 15-20% of children and adolescents in the U.S. are obese. Obesity increases the risk of many chronic diseases in children and adults. Among those diseases, nonalcoholic fatty liver disease (NAFLD) and nonalcoholic steatohepatitis (NASH) are rapidly becoming the most common cause of chronic liver diseases due to an increase in the prevalence of obesity.

NAFLD is the most common chronic liver disease and affects more than 25% of the population in worldwide. NAFLD includes a wide range of clinical conditions from hepatic steatosis, NASH which shows hepatocytes damage and lobular inflammation in association with fibrosis development, and cirrhosis may lead to liver failure or hepatocellular carcinoma. Patients with NAFLD, particular those with NASH, encompasses a cardiometabolic comorbidities includes type 2 diabetes, obesity, metabolic syndrome, and dyslipidemia, which is a leading cause of death. Although the global burden of NAFLD and substantial efforts in therapeutic development, no drug or therapy target has been approved by US Food and Drug Administration (FDA). (Angulo 2002 New England Journal of Medicine 346, 1221-1231; Koo, 2013 Clinical and Molecular Hepatology 19, 210; Sheth, et al. 1997 Annals of Internal Medicine 126, 137-145; Targher, et al. 2013 The Journal of Clinical Endocrinology & Metabolism 98, 483-495; Dietrich, et al. 2014 Best Practice & Research Clinical Gastroenterology 28, 637-653; Charlton 2009 Liver transplantation 15, S83-S89; Sumida, et al. 2018 Journal of Gastroenterology 53, 362-376; Friedman, et al. 2018 Nature Medicine 24, 908-922.)

Protein kinase D (PKD) is a family of serine/threonine protein kinases, which regulate many important cellular functions, including Golgi fragmentation, cell proliferation, apoptosis, and cell migration. There are three PKD isoforms (PKD1, PKD2, and PKD3), encoded by different genes Prkd1, Prkd2, and Prkd3, respectively. PKD isoforms have different expression patterns and functions depending on cell types and external signal stimuli. PKD1 and PKD2 have been implicated in cardiac hypertrophy and T-cell activation, respectively. However, up-to-date knowledge of the physiological function of PKD3 is very limited. Recently we have demonstrated that PKD3 plays a critical role in liver, which maintain the normal liver function and prevent activation of macrophages to induce liver fibrosis. M1/M2 polarization of macrophages controls development of liver disease after liver injury. M1 macrophages are functionally pro-inflammatory and antimicrobial, while M2 macrophages are anti-inflammatory. Macrophage phenotypic changes and activation are critically involved in the pathogenesis of metabolic disorders such as obesity, NAFLD and NASH, which are still no effective therapies available. Therefore, there is a strong rationale to further investigate the potential role of PKD3 in the development of obesity, NAFLD and NASH and ask whether PKD3 could be a new molecular target for effective therapeutics. (Rozengurt, et al. 2005 Journal of Biological Chemistry 280, 13205-13208; Jamora, et al. 1999 Cell 98, 59-68; Wong, et al. 2005 Journal of Biological Chemistry 280, 33262-33269; Zhang, et al. 2005 Journal of Biological Chemistry 280, 19036-19044; Peterburs, et al. 2009 Cancer Research 69, 5634-5638; Rykx, et al. 2003 FEBS Letters 546, 81-86; Li, et al. 2011 Journal of Biological Chemistry 286, 40782-40791; Navarro, et al. 2014 Science Signaling 7, ra99-ra99; Zhang, et al. 2013 Comprehensive Physiology 3, 785-797; Younossi, et al. 2018 Nature Reviews Gastroenterology & Hepatology 15, 11-20.)

Although the extensive studies have been carried out over the past several decades, the molecular targets for the treatment of obesity, and obesity-associated NAFLD and NASH still large elusive. There exists an ongoing and urgent need for novel therapeutic compositions and treatment methods for obesity, NAFLD and NASH and related diseases that offer improved clinical outcome with reduced side effects.

### SUMMARY OF THE INVENTION

Disclosed herein are novel therapeutic compositions and methods for treatment of metabolic diseases that exploit genetic and pharmacological inhibition of Protein Kinase D3 (PKD3, gene name Prkd3). As demonstrated by in vivo studies using mouse models, therapeutic compositions and treatment methods of the invention achieve significant reduction and/or reversion of various risk factors associated with metabolic symptom, such as obesity, NAFLD and NASH. An exemplary PKD3 inhibitor, CRT0066101, is shown herein to effectively treat metabolic syndrome and many of its underlining or associated diseases and conditions.

In particular, PKD3 inhibition is identified as beneficial therapeutic approach to metabolic diseases, such as obesity, NAFLD and NASH. Using genomic and pharmacological method to limit NAFLD development in mice, we provide evidence for a defense role of PKD3 in NAFLD and NASH. In a search for potential PKD3 inhibitor therapies for NASH, we identified CRT 0066101 (PKD3 inhibitor) and PKD3-sh-RNA-AAV has highly effects and potently protecting mice from diet-induced NASH.

In one aspect, the invention generally relates to a method for treating a metabolic symptom, or a related disease or condition thereof. The method comprises administering to a subject in need thereof
a compound of Formula (I)

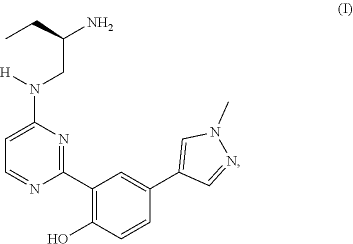 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a metabolic symptom, or a related disease or condition thereof, in a mammal, including a human.

In another aspect, the invention generally relates to a method for treating a cardiovascular disease, or a related disease or condition thereof. The method comprises administering to a subject in need thereof
a compound of Formula (I)

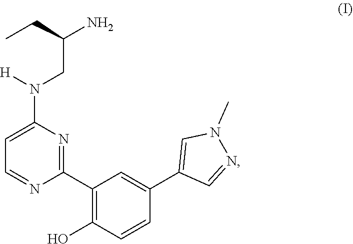 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a cardiovascular disease, or a related disease or condition thereof, in a mammal, including a human.

In yet another aspect, the invention generally relates to a pharmaceutical composition. The pharmaceutical composition comprises
a compound of Formula (I)

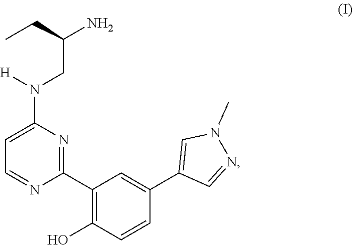 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a metabolic symptom, or a related disease or condition thereof, in a mammal, including a human, and a pharmaceutically acceptable carrier.

In yet another aspect, the invention generally relates to a pharmaceutical composition. The pharmaceutical composition comprises
a compound of Formula (I)

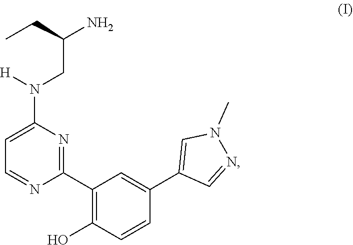 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a cardiovascular disease, or a related disease or condition thereof, in a mammal, including a human, and a pharmaceutically acceptable carrier.

In yet another aspect, the invention generally relates to a unit dosage form comprising the pharmaceutical composition disclosed herein.

In yet another aspect, the invention generally relates to a method for inhibiting protein kinase D3 (PKD3). The method comprises administering to a subject in need thereof
a compound of Formula (I)

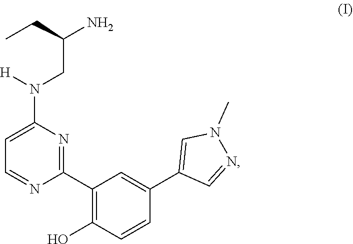 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

and a pharmaceutically acceptable carrier.

### BRIEF DESCRIPTION OF THE DRAWINGS

FIG. 1. Heterozygote deficiency of PKD3 alleviated body weight gain and NAFLD phenotypes in C57BL/6J mice fed with high fat diet (HFD). (A) Body weight growth curve of HFD-fed wild-type (WT) control mice and heterozygous PKD3+/− mutant mice under C57BL/6J background. (B) Representative image of WT and PKD3^{+/-} mice fed with HFD. (C) Body weight. (D) Food intake. (E) Representative image of liver morphology. (F) Histology of liver tissues: H/E staining, Sirius Red staining and Oil Red O staining (scale bars, 50 μm). (G) Histology of fat tissues, H/E staining (scale bars, 50 μm). (H) % of fat weight. (I) Insulin tolerance test (ITT). After 12 h fasting, mice were received insulin via i.p. (5 U/g body weight) and then the levels of blood glucose were measured at the indicated times. (J) Oral glucose tolerance test (OGTT). After 12 h fasting, mice were orally received glucose (2 mg/g body weight) and then the levels of blood glucose were measured at the indicated times. (K) Heatmap of gene profiles in mouse liver tissues from HFD-fed WT mice and PKD3^{+/-} mice.

FIG. 2. The PKD3 inhibitor CRT0066101 reduced bodyweight and NAFLD phenotypes in high fat diet (HFD)-fed C57BL/6J mice. (A) Body weight growth curve of HFD-fed C57BL/6J mice treated (oral gavage) with the vehicle (H₂O) or the PKD3 inhibitor CRT0066101. (B) Body weight growth curve of HFD-fed C57BL/6J after stopping the treatment with the vehicle or the PKD3 inhibitor. (C) Representative image of HFD-fed C57BL/6J mice treated with the vehicle or the PKD3 inhibitor. (D) Body weight. (E) Food intake. (F) Liver weight. (G) Histology of liver tissues (scale bars, 50 μm). (H) The levels of serum aspartate aminotransferase (AST). (I) The levels of serum alanine aminotransferase (ALT). (J) The running distance of physical exercise. (K) Histology of fat tissues (scale bars, 50 μm). (L-N) % of fat weight. (O) Insulin tolerance test (ITT), n=5-8. (P) Oral glucose tolerance test (OGTT), n=5-8. (Q) The levels of random bold glucose. (Q) The levels of fasting blood glucose.

FIG. 3. The PKD3 inhibitor CRT0066101 reduced bodyweight and NAFLD phenotypes in ApoE^{-/-} mice fed with Western diet (WD). (A) Body weight growth curve of WD-fed ApoE^{-/-} mice treated (oral gavage) with the vehicle (H₂O) or the PKD3 inhibitor CRT0066101. (B) Body weight. (C) Food intake. (D) Representative image of liver and spleen morphology. (E) Liver weight. (F) Histology of liver tissues (scale bars, 50 μM). (G) Serum. (H) Histology of fat tissues (scale bars, 50 μm). (I-K) % of fat weight. (L) Heatmap of gene profiles in mouse liver tissues from WD-fed ApoE^{-/-} mice treated with the vehicle or the PKD3 inhibitor.

FIG. 4. Heterozygote deficiency of PKD3 mitigated body weight and NASH phenotypes when the mice fed with high fat high sucrose diet (hereafter referred NASH diet). (A) Body weight growth curve of control WT mice and heterozygous PKD3^{+/-} mutant mice fed with NASH diet. (B) Representative image of WT mice and PKD3^{+/-} mice fed with NASH diet. (C) Body weight. (D) Representative image of liver morphology. (E) Liver weight. (F) Blood glucose. (G) Representative image of fat tissues. (H) % of fat weight.

FIG. 5. The PKD3 inhibitor CRT0066101 reduced bodyweight and NASH in C57 BL/6NJ mice fed with high fat high sucrose diet (hereafter referred NASH diet). (A) Histology of liver tissues from the mice fed with NASH diet (hereafter referred as NASH mice). (B) Body weight curve of NASH mice treated (oral gavage) with the vehicle (H₂O) or the PKD3 inhibitor CRT0066101. (C) Representative image of NASH mice treated with vehicle or the PKD3 inhibitor. (D) Body weight. (E) Representative images of liver morphology. (F) Liver weight. (G) Histology of liver tissues (scale bars, 50 μm), (G) Histology of fat tissues (scale bars, 50 μm), (H) Blood glucose. (I) Representative image of fat tissues. (J) Histology of fat tissues (scale bars, 50 μm). (K) % of fat weight. (L) Heatmap of gene profiles in mouse liver tissues from NASH mice treated with the vehicle or the PKD3 inhibitor.

FIG. 6. PKD3-siRNA-AAV treatment decreased body weight and NAFLD in ApoE^{-/-} mice injected with PKD3-siRNA-AAV on Western diet (WD)-fed conditions. (A) Body weight growth curve of WD-fed ApoE^{-/-} mice injected with control-siRNA-AAV and PKD3-siRNA-AAV. (B) Representative image of WD-fed ApoE^{-/-} mice injected with control-siRNA-AAV and PKD3-siRNA-AAV. (C) Body weight. (D) Liver weight. (E) Histology of liver tissues (scale bars, 50 μm). (F-I) % of fat weight. (G) Histology of fat tissues (scale bars, 50 μm).

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

As used herein, the term “administering” refers to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Suitable routes of administration for a particular patient will depend on the nature and severity of the disease or condition being treated or the nature of the therapy being used and on the nature of the active compound.

Administration may be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies (e.g., anti-cancer agent or chemotherapeutic).

The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, the terms “disease,” “condition,” and “disorder” are used interchangeably herein and refer to a state of being or health status of a patient or subject capable of being treated with a compound, pharmaceutical composition, or method provided herein.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “inhibition,” “inhibit” and “inhibiting” and the like in reference to a biological target (e.g., EGFR) inhibitor interaction refers to negatively affecting (e.g., decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, a “pharmaceutically acceptable form” of a disclosed compound includes, but is not limited to, pharmaceutically acceptable salts, esters, hydrates, solvates, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate.” Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

Prodrug forms often offer advantages of solubility, tissue compatibility, or delayed release in the mammalian organism. (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having a carbonate, carbamate, amide or alkyl ester moiety covalently bonded to any of the above substituents disclosed herein.

Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “prodrug” (or “pro-drug”) refers to a pharmacological derivative of a parent drug molecule that requires biotransformation, either spontaneous or enzymatic, within the organism to release the active drug. Such prodrugs are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism. (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient's age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “high dosage” is meant at least 5% (e.g., at least 10%, 20%, 50%, 100%, 200%, or even 300%) more than the highest standard recommended dosage of a particular compound for treatment of any human disease or condition.

### DETAILED DESCRIPTION OF THE INVENTION

The invention provides novel therapeutic compositions and treatment methods for metabolic symptom. In particular, using PKD3 knockout mouse models, PKD3 pharmacological inhibitor and adeno-associated virus (AAV)-carried PKD3 small interference RNA (siRNA) approaches, it was discovered that the inhibition of PKD3 in mice ameliorated the development and progression of obesity, NAFLD and NASH in several preclinical mouse models of metabolic disorders induced by high fat diet (HFD, which promotes obesity ad NAFLD), high cholesterol Western-type diet (WD, which promotes atherosclerosis and NAFLD), and high fat and high sucrose diet (HFHSD, which promotes obesity and NASH), respectively.

As disclosed herein, genetical and pharmacological inhibition of protein kinase D3 (PKD3) mitigates obesity, NAFLD and NASH in the preclinical mouse models of diet-induced metabolic syndrome. First, heterozygous PKD3 knockout mutant mice fed with high fat diet (HFD, which promotes obesity and NAFLD) displayed a significant decline of HFD-induced increase of obesity weight, fat tissues, fatty liver (NAFLD), liver inflammation and liver fibrosis (NASH characteristics). Similarly, when PKD3 heterozygous mutant mice were challenged with high cholesterol Western-type diet (WD, which promotes atherosclerosis and NAFLD), or high fat and high sucrose diet (HFHSD, which promotes obesity and NASH), the PKD3 mutant mice exhibited a markedly decrease of WD-induced NAFLD and HFHSD-induced obesity and NASH. Moreover, we found that small molecule PKD3 inhibitor CRT0066101 and adeno-associated virus (AAV)-carried PKD3 small interference RNA (siRNA) promotes the regression of various diets-induced obesity, atherosclerosis, NAFLD and NASH phenotypes in mice challenged with HFD, WD, and HFHSD, indicating that CRT0066101 and AAV-PKD3-siRNA have potent therapeutic effects on obesity, atherosclerosis, NAFLD and NASH.

Mechanistically, the PKD3-dependent transcriptomics were explored using RNA-sequencing technologies. It was found that PKD3 depletion or inhibition profoundly influences macrophage phenotypes and activation, which plays a critical role in the pathogenesis of various metabolic disorders. Collectively, the present disclosure has for the first time demonstrated that PKD3 plays an essential role in the development of diet-induced obesity and obesity-associated metabolic syndrome. The proof-of-concept preclinical studies have also revealed that the molecular and pharmacological inhibitions of PKD3 could be utilized as effective therapies for combating obesity, NAFLD and NASH, thus warranting future clinical evaluation.

An PKD3 inhibitor, having the following structure,

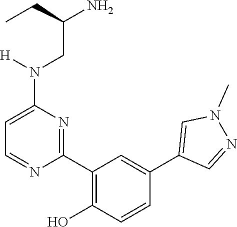 [SMILES: CC[C@@H](N)CNc1ccnc(-c2cc(-c3cnn(C)c3)ccc2O)n1]

or a pharmaceutically acceptable salt, ester or pro-drug thereof, is demonstrated herein to effectively reduce or reverse many of the diseases associated with metabolic syndrome, such as obesity, insulin resistance, diabetes, atherosclerosis, obesity-associated heart failure and fatty liver.

In one aspect, the invention generally relates to a method for treating a metabolic symptom, or a related disease or condition thereof. The method comprises administering to a subject in need thereof
a compound of Formula (I)

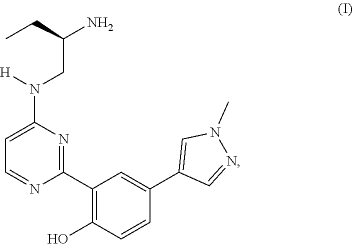 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a metabolic symptom, or a related disease or condition thereof, in a mammal, including a human.

In certain embodiments, the metabolic symptom, or a related disease or condition thereof, is obesity. The term “obesity” as used herein refers to a medical condition in which excess body fat has accumulated to an extent that it may have a negative effect on health. People are generally considered obese when their body mass index (BMI), a measurement obtained by dividing a person's weight by the square of the person's height, is over 30 kg/m².

In certain embodiments, the metabolic symptom, or a related disease or condition thereof, is NAFLD.

In certain embodiments, the metabolic symptom, or a related disease or condition thereof, is NASH.

In certain embodiments, the metabolic symptom, or a related disease or condition thereof, is diabetes.

In certain embodiments, the metabolic symptom, or a related disease or condition thereof, is insulin insensitivity or insulin resistance.

In certain embodiments, the metabolic symptom, or a related disease or condition thereof, is fatty liver.

In certain embodiments, the metabolic symptom, or a related disease or condition thereof, is hepatic steatosis.

In another aspect, the invention generally relates to a method for treating a cardiovascular disease, or a related disease or condition thereof. The method comprises administering to a subject in need thereof
a compound of Formula (I)

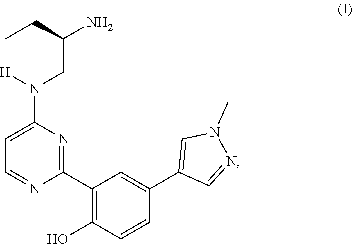 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a cardiovascular disease, or a related disease or condition thereof, in a mammal, including a human.

In certain embodiments, the cardiovascular disease is atherosclerosis.

In certain embodiments, the cardiovascular disease is cardiac hypertrophy.

In certain embodiments, the cardiovascular disease is heart failure.

In certain embodiments, the cardiac hypertrophy or heart failure is induced by obesity.

In certain embodiments, the compound is in the form of an acid-addition salt.

In certain embodiments, the compound is in the form of a HCl salt.

In certain embodiments, the compound is administered orally, intravenously, intramuscularly, or subcutaneously. In certain embodiments, the compound is administered orally.

In certain embodiments, the method of the invention further comprises co-administering to the subject a second therapeutic agent that treats one or more of the risk factors of metabolic symptom selected from the group consisting of central obesity, high blood pressure, elevated fasting plasma glucose, high serum triglycerides and low high-density cholesterol levels.

In certain embodiments, the method of the invention further comprises co-administering to the subject a second therapeutic agent that treats one or more of conditions associated with metabolic symptom selected from the group consisting of atherosclerosis, heart failure, stroke, insulin resistance, type 2 diabetes mellitus, fatty liver and cirrhosis.

In yet another aspect, the invention generally relates to a pharmaceutical composition. The pharmaceutical composition comprises
a compound of Formula (I)

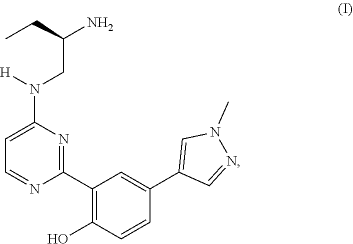 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a metabolic symptom, or a related disease or condition thereof, in a mammal, including a human, and a pharmaceutically acceptable carrier.

In certain embodiments, the metabolic symptom, or a related disease or condition thereof, is obesity.

In certain embodiments, the metabolic symptom, or a related disease or condition thereof, is NAFLD.

In certain embodiments, the metabolic symptom, or a related disease or condition thereof, is NASH.

In certain embodiments, the metabolic symptom, or a related disease or condition thereof, is diabetes.

In certain embodiments, the metabolic symptom, or a related disease or condition thereof, is insulin insensitivity or insulin resistance.

In certain embodiments, the metabolic symptom, or a related disease or condition thereof, is fatty liver.

In certain embodiments, the metabolic symptom, or a related disease or condition thereof, is hepatic steatosis.

In yet another aspect, the invention generally relates to a pharmaceutical composition. The pharmaceutical composition comprises
a compound of Formula (I)

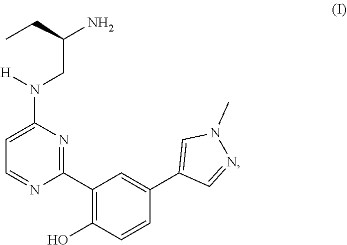 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

in an amount effective in the treatment of a cardiovascular disease, or a related disease or condition thereof, in a mammal, including a human, and a pharmaceutically acceptable carrier.

In certain embodiments, the cardiovascular disease is atherosclerosis.

In certain embodiments, the cardiovascular disease is cardiac hypertrophy.

In certain embodiments, the cardiovascular disease is heart failure.

In certain embodiments, the cardiac hypertrophy or heart failure is induced by obesity.

In certain embodiments, the pharmaceutical composition is suitable for one or more of oral administration, intravenous, intramuscular, and subcutaneous administration. In certain embodiments, the pharmaceutical composition is suitable for oral administration

In yet another aspect, the invention generally relates to a unit dosage form comprising the pharmaceutical composition disclosed herein.

In yet another aspect, the invention generally relates to a method for inhibiting protein kinase D3 (PKD3). The method comprises administering to a subject in need thereof
a compound of Formula (I)

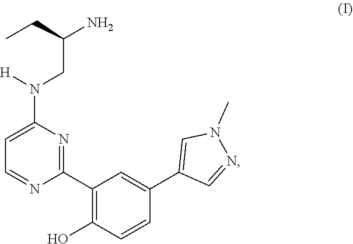 [SMILES: CC[C@@H](N)CNc1ccnc(-c2cc(-c3cnn(C)c3)ccc2O)n1]

or a pharmaceutically acceptable salt, ester or pro-drug thereof, or
a nucleic acid selected from the group consisting of:

(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,

and a pharmaceutically acceptable carrier.

In certain embodiments, the method further comprises co-administering to the subject a second therapeutic agent that treats one or more of the risk factors of metabolic symptom selected from the group consisting of central obesity, high blood pressure, elevated fasting plasma glucose, high serum triglycerides and low high-density cholesterol levels.

In certain embodiments, the method further comprises co-administering to the subject a second therapeutic agent that treats one or more of conditions associated with metabolic symptom selected from the group consisting of obesity, atherosclerosis, heart failure, stroke, insulin resistance, type 2 diabetes mellitus, fatty liver and cirrhosis.

In certain embodiments, the one or more of conditions is obesity. In certain embodiments, is NAFLD.

In certain embodiments, the one or more of conditions is obesity. In certain embodiments, is NASH.

The present disclosure demonstrates that the PKD3 in liver is associated with suppression of hepatic relative genes. In particular, a marked suppression of PKD3 attenuated NAFLD and NASH in murine was observed, which showed that PKD3 expression inversely correlated with hepatic fat and body fat. Although our previous study showed that deletion of PKD3 in mouse induce liver fibrosis, our work underscores a protection role for PKD3 in NAFLD and NASH. The role of PKD3 in NASH firstly evaluated by us. Using genomic method, we generated PKD3+/− mice that present attenuated NASH after 6 weeks on NASH diet. We identified inhibitor of PKD3 by drug block steotohepatitis and fibrosis in PKD3+/− mice, supporting the premise that PKD3 plays a protection role in NASH.

Dietary approaches were further applied to limit PKD3 function and used C57 BL/6J mice fed HFD, ApoE−/− mice fed WD and C57 BL/6NJ mice fed NASH diet with or without PKD inhibitor, allowing the protected study of obesity, body fat, glucose tolerance, liver fibrosis and hepatic steatosis (HS). The reduced body weight, fat accumulation fibrosis, and HS observed in mice fed HFD, WD or NASH diet are supported by previous reports in which accelerated fat loss, improved glucose tolerance, reduced fibrosis, or HS in three different rodent models. These findings suggest a protection role for PKD3 in NAFLD and, simultaneously, constitute a strong rationale to examine its therapeutic potential. Here in a thorough investigation using a long-term dietary model of advanced NAFLD featuring co-existence of steatohepatitis and fibrosis, which better mimics the human disease, we document causative effects of PKD3.

In previous studies, loss of PKD3 induce liver fibrosis in mice[17]. To avoid liver fibrosis spontaneously development in current study, we applied PKD3 Heterozygote mice and oral administration of low dose PKD3 inhibitor to the mice. We report here protective effects in NASH model at a lower dose of 100 ug/10 g per day. Our findings suggest that a minimum of 10 mg/kg per day of PKD3 inhibitor may have beneficial effects in human. Although the potential benefits of PKD3 inhibitor administration to patients with NAFLD and evaluation of the doses had not be reported yet, the preclinical study have confirmed that the PKD3 inhibitor is potential therapeutic target in the future, further clinical trial study should be try.

In a search for PKD3-siRNA-AAV target more potent than small molecular compound of PKD3 effects, we tested PKD3-siRNA-AAV in WD-induced NAFLD, we reported to decrease body weight, promote body fat loss, improve glucose tolerance and reduce hepatic steatosis in mice. Applying various dietary models, our lab uncovered potent PKD3-siRNA-AAV effects for the NASH. Here, we show that CRT0066101 also diminish NASH and NAFLD in murine models. In addition, profound benefits were noted with the single dose of PKD3-siRNA-AAV. Treatment with PKD3-siRNA-AAV one time prevented NASH diet-induced alternations in body composition and reduced liver NAS and liver fibrosis, indicating that PKD3-siRNA-AAV was more potent than equivalent amounts of CRT0066101 in protecting against diet-induced NASH.

The present invention provides translational potential for the clinical management of NAFLD, currently without approved treatments. In humans, although with small sample sizes and not including patients with NASH, PKD3 inhibitor treatments were shown to improve liver function, reduced body weight and fat accumulation. Furthermore, PKD3-sh-RNA-AAV is well tolerated and safe than even at PKD3 inhibitor.

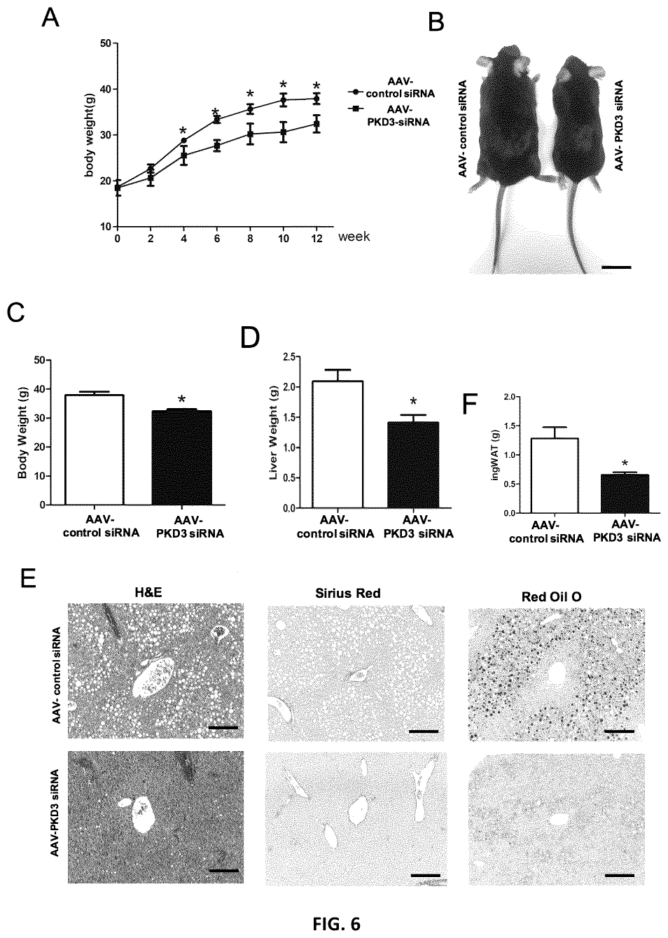

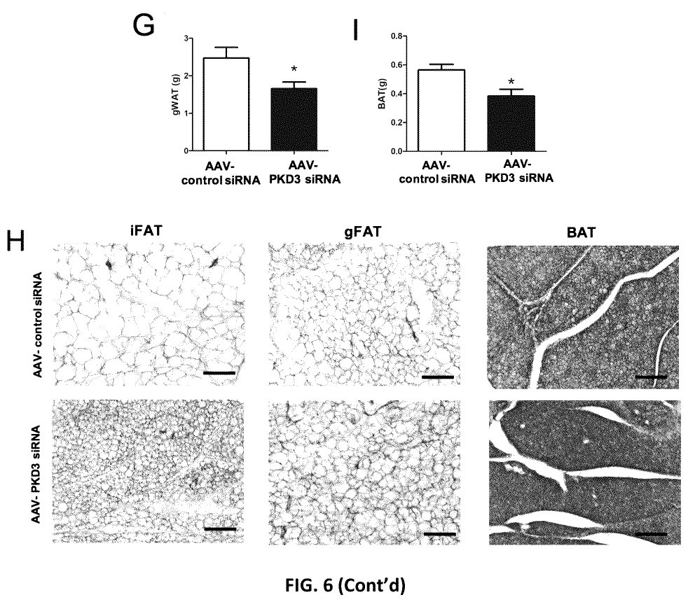

AAV-PKD3 siRNA/shRNA are disclosed herein that can be therapeutic reagents in addition to the small molecule inhibitors. For example, AAV8-PKD3 siRNAs were used to inhibit PKD3 and achieved significant therapeutic effects on obesity and NAFLD (FIG. 6, AAV8-PKD3-siRNAs was injected to mice after NAFLD has been established). The mouse sequences of PKD3 siRNA used for mouse studies were:

Target a-239:
(SEQ ID NO: 5)
TGTCTCTCTCTGCTGTCAAAGACCTCGTG
Target b-817:
(SEQ ID NO: 6)
ACATTTGCTGTCCACTCTTATGGCCGCCC
Target c-1795:
(SEQ ID NO: 7)
GGAAGGGATGTGGCTATTAAAGTGATTGA
Target d-2281:
(SEQ ID NO: 8)
TCGGTGGGCGTCATCGTTTATGTGAGCCT

These mouse PKD3 siRNA sequences correspond to human PRKD3 siRNA sequences as follows:

Human PKD3 siRNA Target A-239:
(SEQ ID NO: 1)
TGTCTTTATCTGCTGTCAAGGATCTTGTG
Human PKD3 siRNA Target B-817:
(SEQ ID NO: 2)
ACATTTGCTGTTCACTCTTACACCCGTCC
Human PKD3 siRNA Target C-1795:
(SEQ ID NO: 3)
GGGAGGGATGTGGCTATTAAAGTAATTGA
Human PKD3 siRNA Target D-2281:
(SEQ ID NO: 4)
TCAGTGGGAGTTATCATCTATGTGAGCCT

As used herein, RNAi active sequences may include “siRNA” and “shRNA” and dsRNA that is processed by nucleases to provide siRNA and/or shRNA. The term “siRNA” refers to a “small interfering RNA” or “small interference RNA” and the term “shRNA” refers to “short hairpin RNA.” RNA interference (RNAi) refers to the process of sequence-specific post-transcriptional gene silencing in a cell or an animal mediated by siRNA and/or shRNA.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The following examples are meant to be illustrative of the practice of the invention, and not limiting in any way.

### EXAMPLES

### Heterozygote Deficiency of PKD3 Alleviated Obesity and NAFLD Phenotypes in C57BL/6J Mice Fed with High Fat Diet

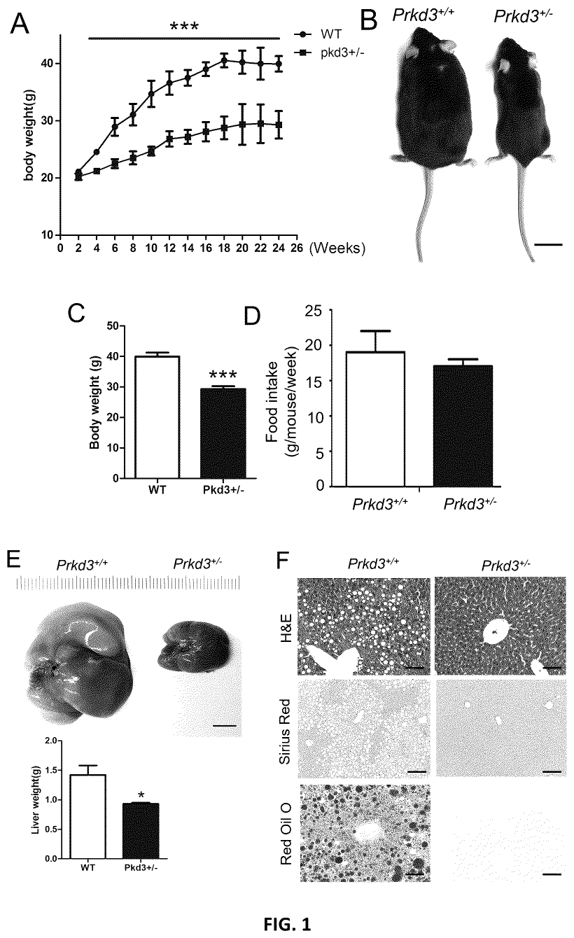

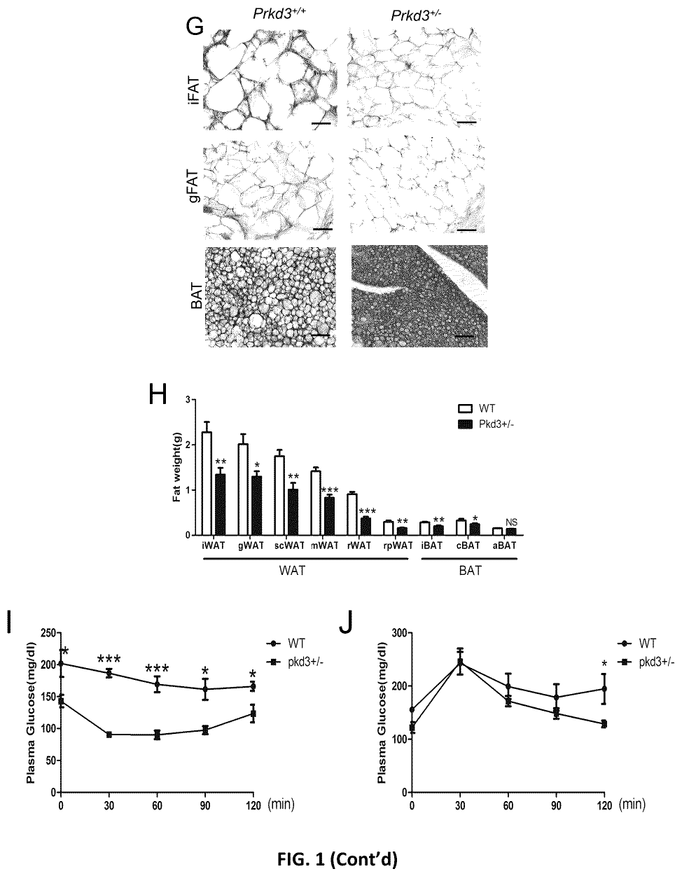

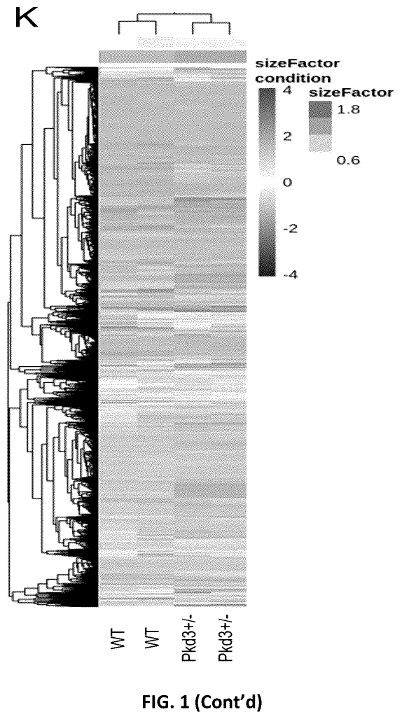

To test whether PKD3 contributes to the development of obesity and NAFLD, we first studied heterozygous PKD3 mutant (PKD3^{+/-}) mice and control PKD3 wild-type (WT) mice with high-fat diet-induced obesity. HFD (Research Diet, D12451) is a rodent diet with 45 kcal % fat, which is well known as the gold standard in high-fat diets for diet-induced obesity research worldwide. Under HFD feeding, the body weight of PKD3^{+/-} mice increased slowly and had significantly statistic differences starting from 4 weeks on HFD feeding when compared with that of WT mice (FIG. 1A). The body weight of PKD3^{+/-} mice was also much less than that of WT mice after 26 weeks on HFD feeding (FIG. 1B-1C). There was no difference of food intake between WT mice and PKD3^{+/-} mice (FIG. 1D). Interestingly, we found that the gross morphology of the livers from PKD3+/− mice looked much better, including smooth liver surface and normal liver color and decreased liver weight, than that of the livers from WT mice (FIG. 1E). We then performed hematoxylin and eosin (H&E) staining to examine tissue morphology, Sirius Red staining to assess fibrosis and Oil Red O (ORO) staining to quantify lipid levels in the liver tissues sections. We found that there was little hepatic ballooning, no liver fibrosis and no hepatic steatosis in the liver tissues from PKD3^{+/-} mice (FIG. 1F), indicating that genetic depletion of PKD3 mitigates HFD-induced NAFLD phenotypes. Furthermore, we isolated various fat tissues from mice and performed the H&E staining. We observed that the fat weight and the size of adipocytes in the fat tissues from PKD3^{+/-} mice was significantly smaller than those from WT mice (FIG. 1G-1H). Insulin tolerance test (ITT) and oral glucose tolerance test (OGTT) demonstrated improved glucose metabolism and insulin sensitivity in PKD3^{+/-} mice (FIG. 1I-IJ). To explore the molecular mechanisms underlying a reduction of HFD-induced obesity and NAFLD in PKD3^{+/-} mice, we performed RNA sequencing (RNA-seq) studies using the liver tissues from WT mice and PKD3^{+/-} mice. The heat map shows a variety of genes was altered in PKD3^{+/-} mice (FIG. 1K). Taken together, our results reveal that genetic PKD3 heterozygous deficiency alleviates body weight gain, fat accumulation, NAFLD, glucose in tolerance and insulin resistance in HFD-induced obesity.

### Pharmacological PKD3 Inhibitor Mitigates Obesity and NAFLD Phenotypes in HFD-Fed C57BL/6J Mice

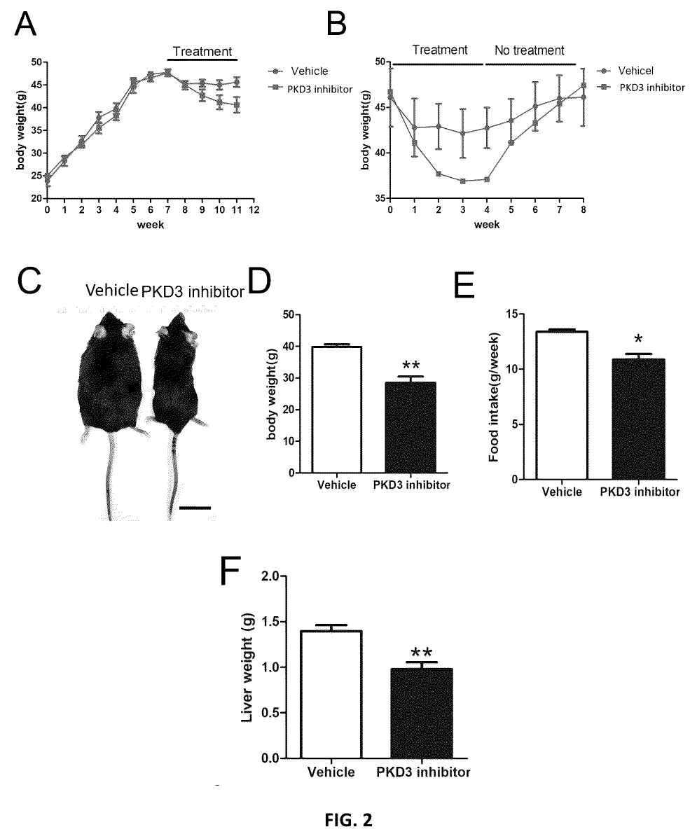

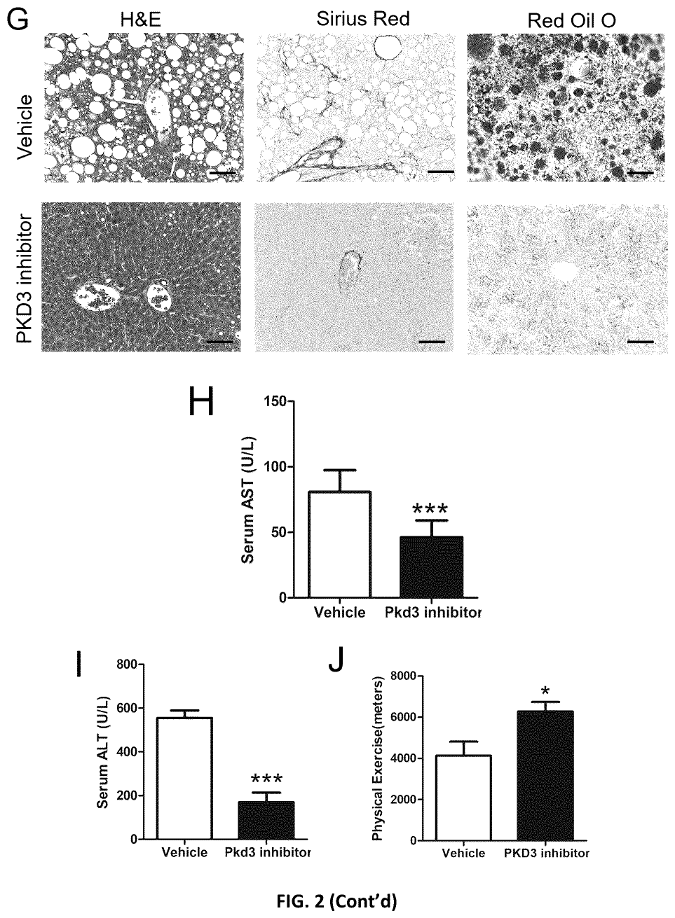

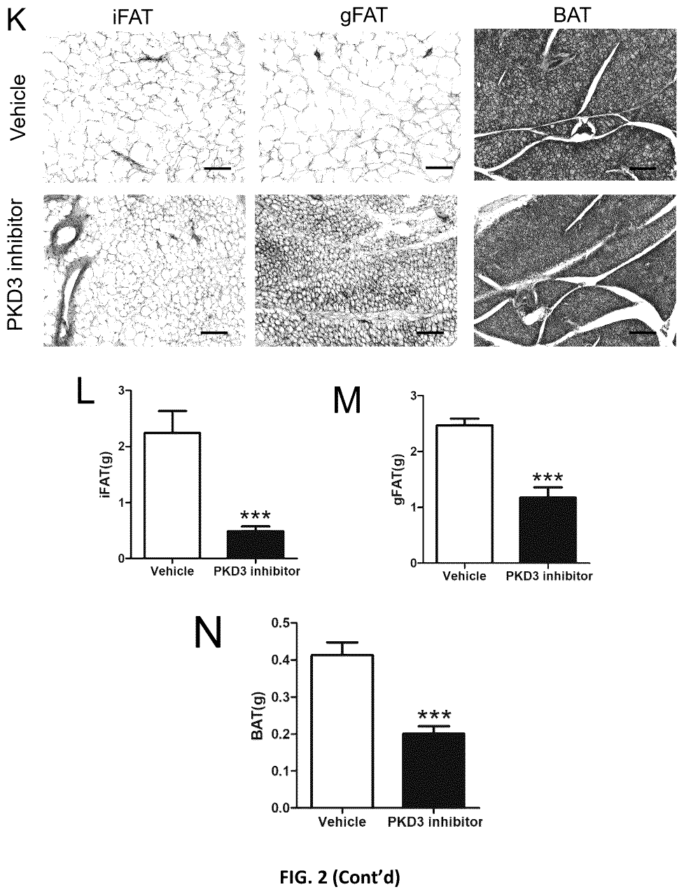

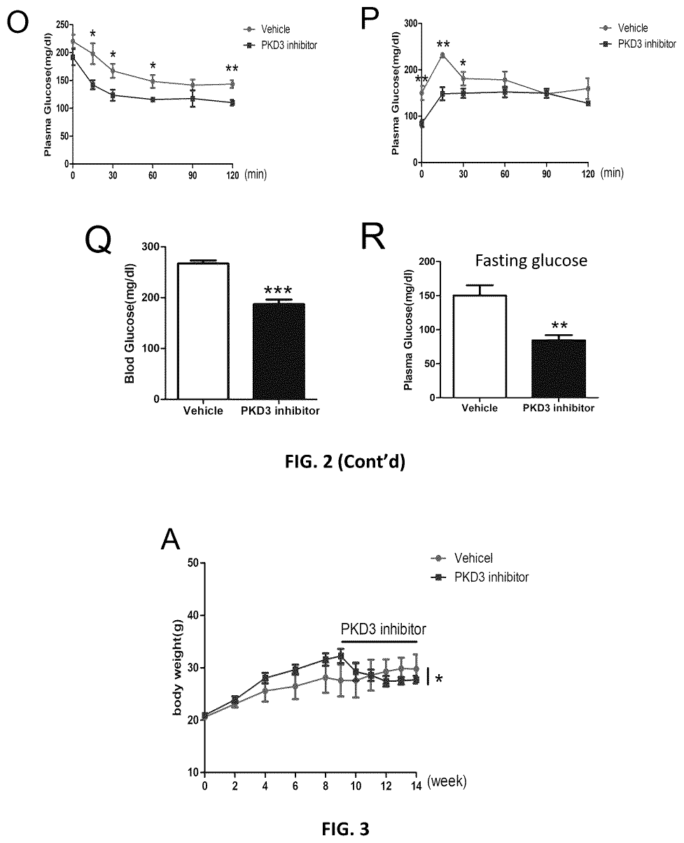

After showing that genetic PKD3 inactivation in mice reduces HFD-induced obesity and NAFLD, we next asked whether the small compounds of the pharmacological PKD3 inhibitor could have a therapeutic potential for the treatment of obesity and NAFLD. One of the potent PKD3 inhibitors is CRT0066101, which selectively inhibits PKD3 with IC₅₀ value of 2 nM. We purchased CRT0066101 from Key Organics (Bedford, Massachusetts, USA). We first development HFD-induced with HFD-induce obesity and NAFLD in C57BL/6J mice and treated them with CRT0066101. Briefly, C57BL/6J adult mice were on HFD for 6 weeks; and we then treated those mice with CRT0066101 by oral gavage (low dose; 10 mg/kg/day) or the vehicle (H₂O) on continue fed HFD conditions. We found that the treatment of CRT0066101 greatly promoted body weight loss of HFD-fed mice (FIG. 2A). After stopping the administration of CRT0066101, mouse body weigh was gradually recovered and reached the same level with the vehicle-treated mice around 7 weeks (FIG. 2B). The mouse fat and liver tissues from the experimental mice were harvested after oral administration one and a half month. At the end-point of CRT0066101 administration, mouse body weight was dramatically decreased in CRT006610-treated mice compared with the vehicle-treated mice (FIG. 2C-2D). The food intake was slightly decreased in CRT0066101-treated mice (FIG. 2E). The liver weight was significantly reduced in CRT0066101-treated mice (FIG. 2F). From the histological studies of the liver tissues from mice, it is clear that the vehicle-treated mice had hepatic ballooning, hepatocyte death, severe liver fibrosis and serious fatty liver, which are the characteristic phenotypes of NASH. In contrast, CRT0066101-treated mice had almost normal liver morphology, little hepatic ballooning and fibrosis as well as lipid deposits (FIG. 2G), indicating that the treatment of CRT0066101 greatly mitigates NASH phenotypes in HFD-induced mice. Moreover, CRT0066101-treated mice had a decreased serum aspartate aminotransferase (AST) and alanine-aminotransferase (ALT) (FIG. 2H-2I), two hallmarks of liver damage. We also found that the running distance of mouse physical exercise on treadmill was increased in CRT0066101-treated mice (FIG. 2J), suggesting a better cardiac function and muscular strength in CRT0066101-treated mice. Furthermore, the size of adipocytes and the weight of fat tissue from CRT0066101-treated mice was considerably diminished (FIG. 2K-2N).

ITT and OGTT analysis showed that the treatment of CRT0066101 improved insulin sensitivity and glucose tolerance in HFD-fed mice (FIG. 2O-2P). The treatment of CRT0066101 also decreased the levels of non-fast blood glucose and fast blood glucose in mice (FIG. 2Q-2R). Collectively, these results provide the in vivo evidence that the oral administration of the small molecule PKD3 inhibitor protects against HFD-induced obesity, NAFLD, NASH and insulin resistance in the mouse model of HFD-induced obesity.

### Pharmacological PKD3 Deletion in Mice with Western Diet Treatment

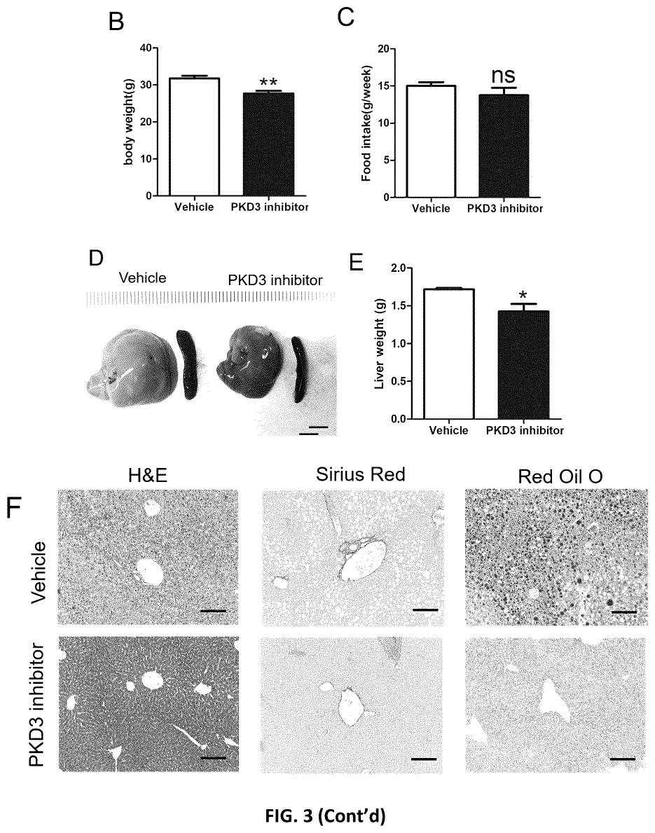

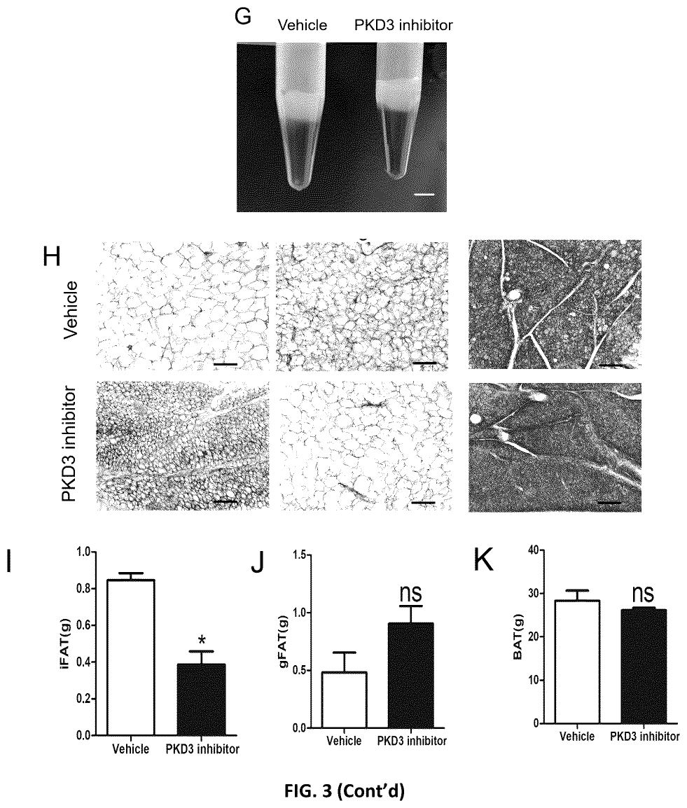

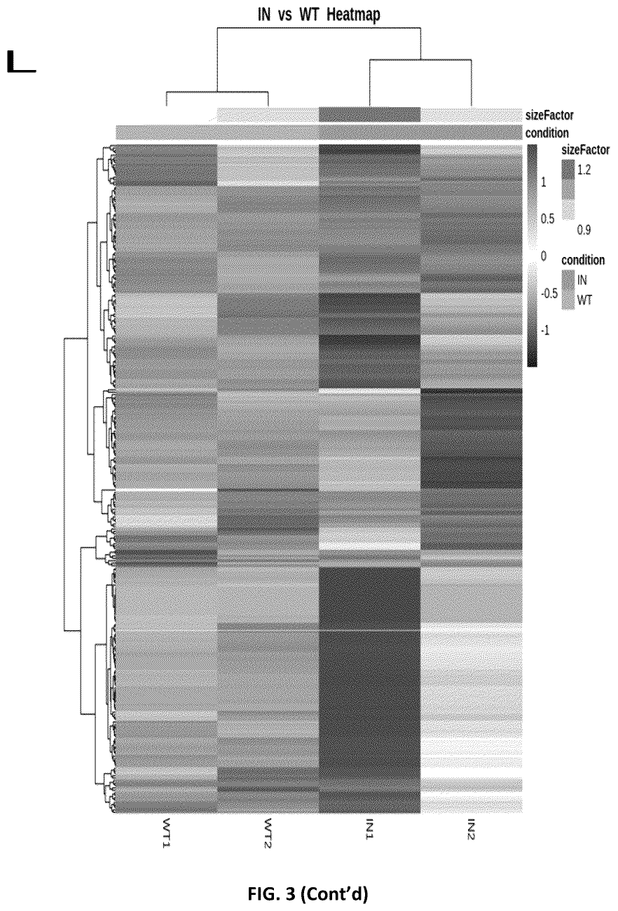

To further test whether PKD3 inhibitor prevents NAFLD development, we first studied ApoE^{-/-} mice with Western diet (WD) induced HS, after 8 weeks on WD, we then treat the mice with PKD3 inhibitor by oral administration (low dose; 100 ug/10 g per day) on continue fed WD condition. The mouse body weight rapidly decreased after PKD3 inhibitor treatment (FIG. 3A). The mouse body weight also decreased at end-point, but the food intake no significant difference in treat or non-treat mice (FIG. 3B-3C). Although the yellowish coloration shown in Vehicle mice, there are not any yellow color in mice with PKD3 treatment (FIG. 3D), and the liver weight was decreased (FIG. 3E). To assess whither a similar pattern was evident in WD-treatment mice. Histological analyses base on H&E and Sirius Red and Oil Red O (ORO) staining confirmed NAFLD activity score (NAS) and fibrosis score was alleviated in mice with PKD3 inhibitor administration (FIG. 3F). Significantly, the hyperlipidemia also decreased in mice with two months PKD3 inhibitor treatment (FIG. 3G). Moreover, the PKD3 fed mice body fat was radically reduced, but the GFAT and BAT no changes in PKD3 treat or non-treat mice (FIG. 3H-3K). To further explore potential mechanisms, we performed RNA sequencing of livers collected at endpoint. Heat maps reveal that the gene expression profile from mice on WD treatment with PKD3 inhibitor, with H₂O controls. These findings indicated that the different diet induced obesity and fatty liver is likely to be reverse by PKDE inhibitor.

### PKD3 Inhibitor Protects Against NASH-Diet Induced NASH in Genomic and Pharmacological Mouse Model

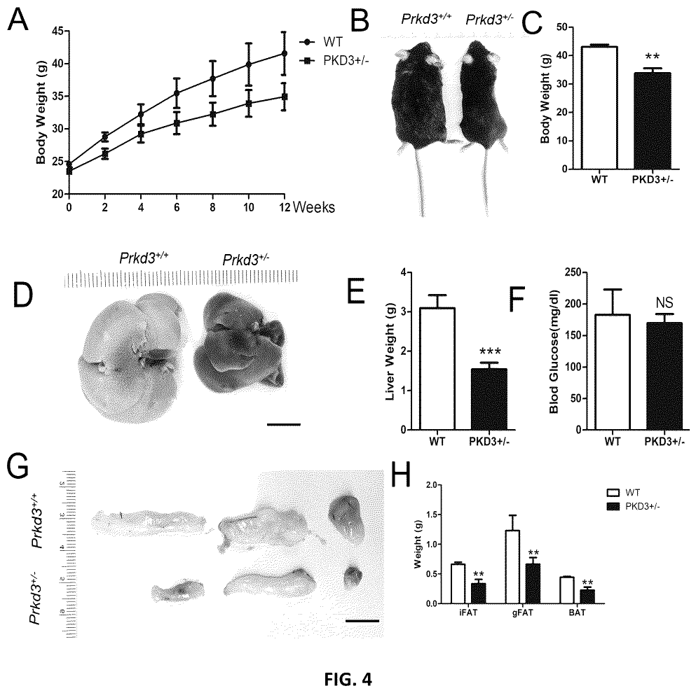

To assess whether a similar phenotype was evident in more severs NAFLD, we performed that PKD3^{+/-} mice fed by 24 weeks on high-fat, high-fructose, high-cholesterol diet (also called NASH diet). The NASH diet D09100310, purchased from Research Diet, is a rodent diet with 40 kcal % Fat (mostly palm oil), 20 kcal % fructose and 2% cholesterol. The growth curve showed that the body weight increased would be prevent on NASH diet fed PKD3^{+/-} mice compared with WT mice (FIG. 4A). The mouse body weight was decreased at endpoint (FIG. 4 B-4C). The liver coloration is redder in PKD3+/− mice (FIG. 4D), liver weight also decreased (FIG. 4E), but the blood glucose not significant changes (FIG. 4F) when compared with WT mice. Base on mouse body weight loss, the body fat also decreased when verses with WT mice, including gFAT, iFAT and BAT (FIG. 4 G-4H).

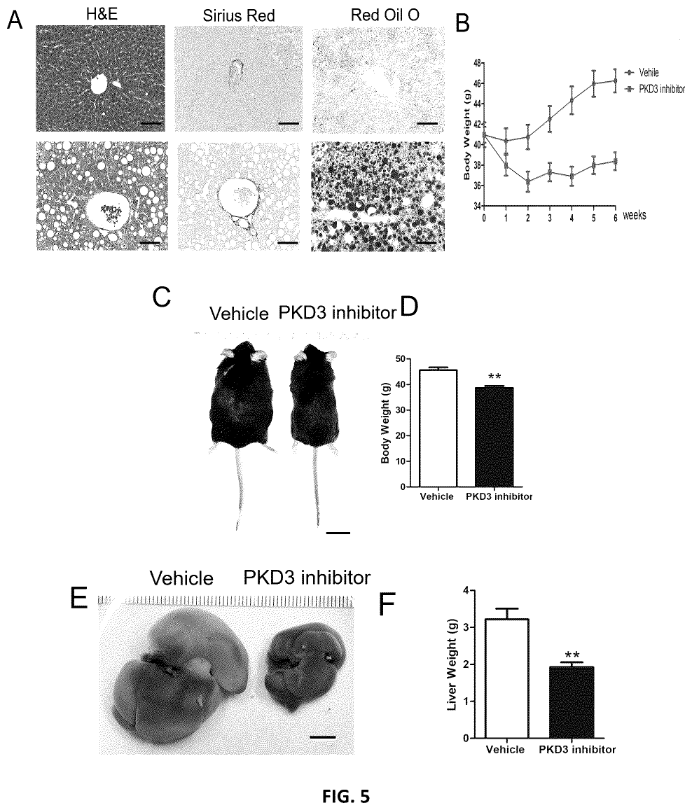

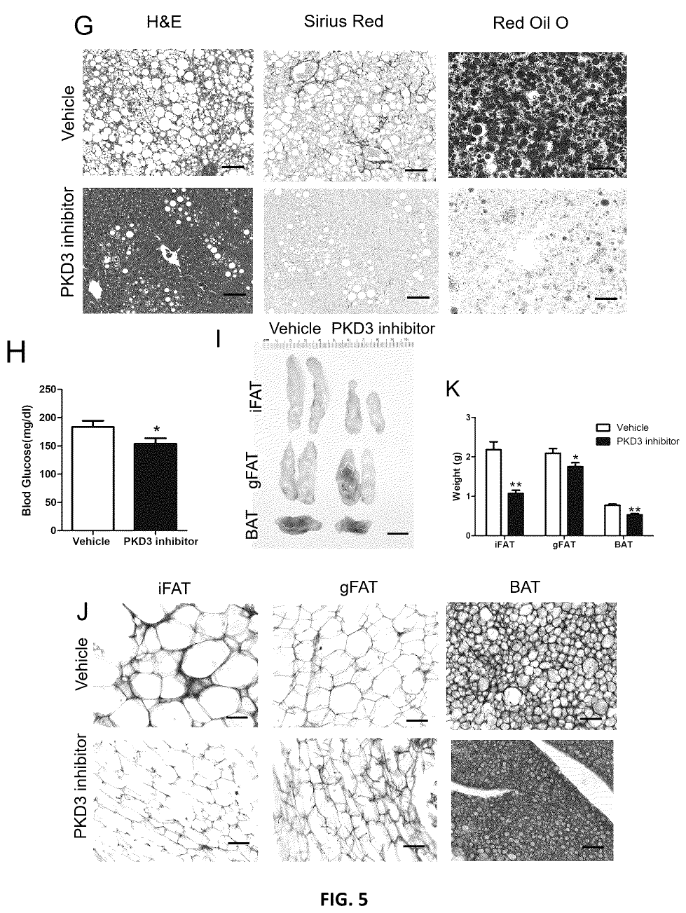

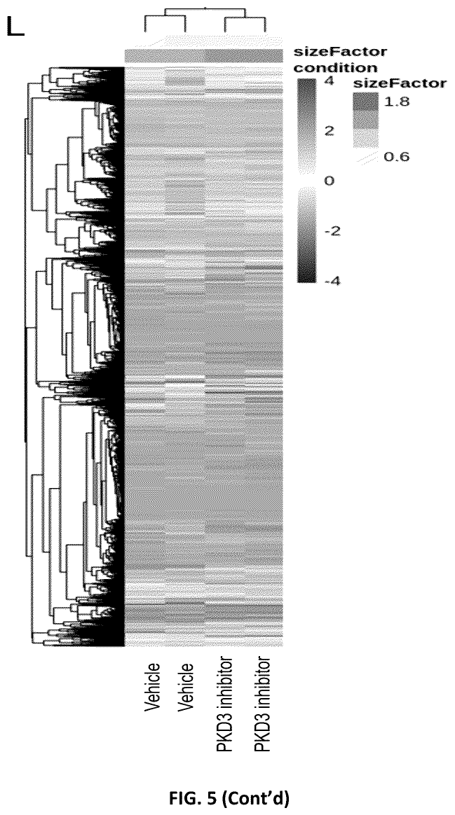

Furtherly, to investigate the therapeutic potential of PKD3 inhibitor against NASH, we devised an experimental approach to model advanced NAFLD. C57BL/6NJ mice fed NASH diet for 12 weeks had increased hepatic steatosis, hepatic ballooning, and early liver fibrosis compared to those fed standard chow diet (CD) (FIG. 5A). After confirming NASH, the rest of the mice were randomized to orally receive PKD3 inhibitor 100 ug/10 g per day or equivalent amounts of H₂O for additional 6 weeks on NASH diet. Body composition analysis revealed decreased body weight in PKD3 inhibitor treatment compared to H₂O treatment (FIG. 5B) in NASH diet feeding. Compared to vehicle mice, the PKD3 inhibitor fed mice body weight was reduced at endpoint (FIG. 5C-5D). Accordingly, the liver surface was more smooth, liver size and weight dramatically decreased after PKD3 inhibitor treatment (FIG. 5E-5F). The sever hepatomegaly induced by NASH diet was markedly attenuated by treatment with PKD3 inhibitor, with NAS, hepatic fibrosis and liver fat significantly decreased by PKD3 inhibitor (FIG. 5G). The nonfasting glucose measurement confirmed the most potent glucose-lowering effect for PKD3 inhibitor (FIG. 5H). The metabolic response to diet-induced obesity involves a shift toward lower fat size and weight, reflecting a reduction in gFAT, iFAT and BAT (FIG. 5I-5K). To explore potential mechanisms by which PKD3 inhibitor treatment protect against diet-induced NASH. We applied RNA sequencing of livers collected at endpoint. Heat map confirmed varied of gene expression was changed (FIG. 5L). Thus, PKD3 treatment reduces NASH diet-induced NASH and liver fibrosis.

### PKD3-siRNA-AAV Reduces Obesity and Fatty Liver Induced by WD-Diet

Based on our findings above together with to show role of PKD3 in genomic and pharmacological condition. We reasoned that PKD3-siRNA-AAV, different methods to target PKD3, also is useful for diet-induced obesity and fatty liver. Thus, we designed PKD3 siRNA to target PKD3 in liver. To test the effects of PKD3 siRNA on diet-induced fatty liver, we fed ApoE^{-/-} mice WD and then injected mice via tail vein with a single dose of PKD3-siRNA-AAV (title 10¹²) or control-siRNA-AAV. The mouse body weight was measurement every two weeks. The curve shows that body weight slightly increased in PKD3-siRNA-AAV injected mice compared to control siRNA-AAV injected mice (FIG. 6A). After 12 weeks, PKD3-siRNA-AAV injected mice had the most potent body weight-lowering (FIG. 6B-6C). Furthermore, liver weight also decreased compared to control mice (FIG. 6D). Histological analyses revealed a reduction of hepatic steatosis in livers from mice treated with PKD3-siRNA-AAV (FIG. 6E). The metabolic analysis to diet-induced obesity including reduced fat weight (FIG. 6F-6I). In conclusion, PKD3 inhibition protect against diet-induced obesity and fatty liver.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The examples herein contain important additional information, exemplification and guidance that can be adapted to the practice of this invention in its various embodiments and equivalents thereof.

1. A method for treating a metabolic symptom, or a related disease or condition thereof, comprising administering to a subject in need thereof
a compound of Formula (I) 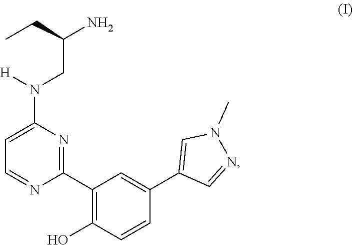 [SMILES: CC[C@@H](N)CNc1ccnc(-c2cc(-c3cnn(C)c3)ccc2O)n1]
or a pharmaceutically acceptable salt, ester or pro-drug thereof, or
a nucleic acid selected from the group consisting of:
(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,
in an amount effective in the treatment of a metabolic symptom, or a related disease or condition thereof, in a mammal, including a human.

2. The method of claim 1, wherein the metabolic symptom, or a related disease or condition thereof, is obesity.

3. The method of claim 1, wherein the metabolic symptom, or a related disease or condition thereof, is nonalcoholic fatty liver disease (NAFLD).

4. The method of claim 1, wherein the metabolic symptom, or a related disease or condition thereof, is nonalcoholic steatohepatitis (NASH)

5. The method of claim 1, wherein the metabolic symptom, or a related disease or condition thereof, is diabetes.

6. The method of claim 1, wherein the metabolic symptom, or a related disease or condition thereof, is insulin insensitivity or insulin resistance.

7. A method for treating a cardiovascular disease, or a related disease or condition thereof, comprising administering to a subject in need thereof
a compound of Formula (I) 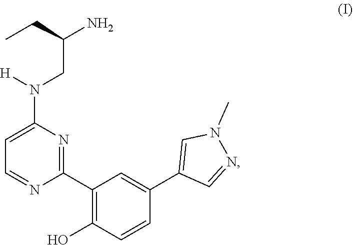 [SMILES: CC[C@@H](N)CNc1ccnc(-c2cc(-c3cnn(C)c3)ccc2O)n1]
or a pharmaceutically acceptable salt, ester or pro-drug thereof, or
a nucleic acid selected from the group consisting of:
(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,
in an amount effective in the treatment of a cardiovascular disease, or a related disease or condition thereof, in a mammal, including a human.

8. The method of claim 7, wherein the cardiovascular disease is atherosclerosis.

9. The method of claim 7, wherein the cardiovascular disease is cardiac hypertrophy.

10. The method of claim 7, wherein the cardiovascular disease is heart failure.

11. The method of claim 7, wherein the cardiac hypertrophy or heart failure is induced by obesity.

12. The method of claim 1, wherein the compound is in the form of an acid-addition salt.

13. The method of claim 7, wherein the compound is in the form of a HCl salt.

14. The method of claim 1, wherein the compound is administered orally, intravenously, intramuscularly, or subcutaneously.

15. A pharmaceutical composition comprising
a compound of Formula (I) 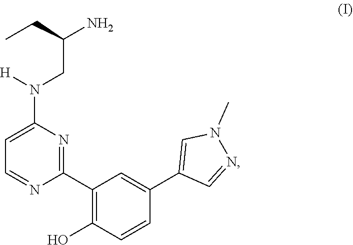 [SMILES: CC[C@@H](N)CNc1ccnc(-c2cc(-c3cnn(C)c3)ccc2O)n1]
or a pharmaceutically acceptable salt, ester or pro-drug thereof, or
a nucleic acid selected from the group consisting of:
(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,
in an amount effective in the treatment of a metabolic symptom or a cardiovascular disease, or a related disease or condition thereof, in a mammal, including a human, and a pharmaceutically acceptable carrier.

16-26. (canceled)

27. A unit dosage form comprising the pharmaceutical composition of claim 15.

28. A method for inhibiting protein kinase D3 (PKD3), comprising administering to a subject in need thereof
a compound of Formula (I) 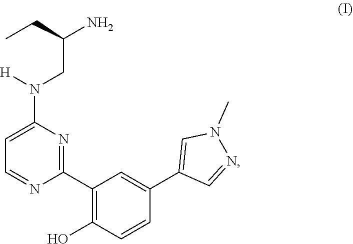 [SMILES: CC[C@@H](N)CNc1ccnc(-c2cc(-c3cnn(C)c3)ccc2O)n1]
or a pharmaceutically acceptable salt, ester or pro-drug thereof, or
a nucleic acid selected from the group consisting of:
(SEQ ID NO: 1)
TGTCTTTATCTGCTGTCAAGGATCTTGTG,
(SEQ ID NO: 2)
ACATTTGCTGTTCACTCTTACACCCGTCC,
(SEQ ID NO: 3)
GGGAGGGATGTGGCTATTAAAGTAATTGA,
and
(SEQ ID NO: 4)
TCAGTGGGAGTTATCATCTATGTGAGCCT,
and a pharmaceutically acceptable carrier.

29. The method of claim 1, further comprising co-administering to the subject a second therapeutic agent that treats one or more of the risk factors of metabolic symptom selected from the group consisting of central obesity, high blood pressure, elevated fasting plasma glucose, high serum triglycerides and low high-density cholesterol levels.

30. The method of claim 1, further comprising co-administering to the subject a second therapeutic agent that treats one or more of conditions associated with metabolic symptom selected from the group consisting of obesity, atherosclerosis, heart failure, stroke, insulin resistance, type 2 diabetes mellitus, fatty liver and cirrhosis.

31. The method of claim 30, wherein the one or more of conditions is obesity.

32. (canceled)

33. (canceled)
